(19) United States
(12) Patent Application Publication
(10) Pub. No.: US 2002/0117175 A1
(43) Pub. Date: Aug. 29, 2002

(54) Thermal vaporizing device for drug delivery

(76) Inventors: S. George Kottayil, Long Grove, IL (US); Peter Anthony Crooks, Lexington, KY (US); David Riggs, Long Grove, IL (US); Ram Murty, Lexington, KY (US)

Correspondence Address: ANTONELLI TERRY STOUT AND KRAUS, SUITE 1800, 1300 NORTH SEVENTEENTH STREET, ARLINGTON, VA, 22209

(21) Appl. No.: 09/984,294

(22) Filed: Oct. 29, 2001

Related U.S. Application Data
(60) Provisional application No. 60/243,404, filed on Oct. 27, 2000.

(51) Int. Cl. A61N 1/30; A61M 11/00; B05D 7/14; A61M 15/00; A61M 16/00; B65D 83/06; F23D 11/00; F23D 14/00

(52) U.S. Cl. 128/203.15; 128/200.14; 128/203.26; 604/19

(57) ABSTRACT

A drug delivery device for delivery of a drug to the lungs of a user includes a tubular housing having a mouth a mouthpiece, a heat source, and a middle section between the mouthpiece and the heat source. The middle section has an chamber containing a plurality of inert inorganic particles which have a drug adsorbed as a coating thereon. The heat source, the plurality of inert inorganic particles in the middle section and the mouthpiece are fluidly connected through an interior passageway of the tubular housing and the distal end section is fluidly connected to outside air. When a user applies suction to the mouthpiece, air from the outside is drawn into the heat source by suction and is heated, and the heated air is drawn into the chamber of the middle section to heat, volatilize and desorb the drug from the inert particles. The volatilized and desorbed drug is then drawn from the chamber of the middle section into the mouthpiece and then into the mouth and lungs of the user.

This application claims priority based on U.S. Provisional Application No. 60/243,404, filed Oct. 27, 2000. The contents of this application are incorporated herein by reference.

### FIELD OF INVENTION

The present invention relates to a thermal vaporizing device suitable for the delivery of volatile drugs to the lung.

### BACKGROUND OF THE INVENTION

Many drugs and pharmacologically active natural products have poor bioavailability when given by the oral route. This may be due to a number of factors, which include high lipophilicity, significant first pass metabolism, poor aqueous solubility or difficulty in formulating an oral dosage form. In addition, for some therapeutic indications, rapid and significant bioavailability is required, which is usually not possible via the oral route, or via other alternative drug delivery systems, such as transdermal and rectal formulations. Also, delivery of such drugs via parenteral injection (i.e., intramuscular, intravenous, or subcutaneous) while providing rapid onset of drug action, has the disadvantage of being an invasive procedure for the patient, requiring professional assistance, and precluding self-medication.

### SUMMARY OF THE INVENTION

It is the object of this invention to provide a device that is capable of vaporizing drugs, such as volatile lipophilic drugs, for rapid delivery to the lung and to the systemic circulation. Lipophilic drugs can be rapidly absorbed from the lungs, and systemic blood levels can be quickly attained. The present invention affords a formulation of the drug that will allow the patient to self-medicate through inhalation of a thermally volatilized drug. In this process, the drug is heated when the device is activated through patient inhalation. The resulting vaporized drug is then inhaled by the patient into the lungs.

It is a further object of the present invention to provide a novel device that when activated by the patient causes a drug substance to be heated, and the resulting drug vapor, representing an aliquot part of the original drug mass, is inhaled by the patient into the lungs.

It is a further object of the present invention to provide a device that can be used for such purposes as the delivery of volatile or moderately volatile drugs and pharmacologically active natural products that require good bioavailability and a rapid onset of action. It is particularly useful for the delivery of such drugs that have low bioavailability via the oral route.

These and other objects are achieved by a drug delivery device for delivery of a drug to the lungs of a user that includes a plurality of inert inorganic particles, the particles having the drug adsorbed thereon, means for heating the plurality of inert inorganic particles to cause the drug to volatilize and become desorbed from the plurality of inert inorganic particles, and means for directing the volatilized and desorbed drug into the lungs of the user.

More particularly, the present invention comprises a tubular housing having a mouth a mouthpiece, a heat source, and a middle section between the mouthpiece and the heat source. The middle section has an chamber containing a plurality of inert inorganic particles which have a drug adsorbed as a coating thereon. The heat source, the plurality of inert inorganic particles in the middle section and the mouthpiece are fluidly connected through an interior passageway of the tubular member and the distal end section is fluidly connected to outside air. When a user applies suction to the mouthpiece, air from the outside is drawn into the heat source by suction and is heated, and the heated air is drawn into the chamber of the middle section to heat, volatilize and desorb the drug from the inert particles. The volatilized and desorbed drug is then drawn from the chamber of the middle section into the mouthpiece and then into the mouth and lungs of the user.

### BRIEF DESCRIPTION OF THE DRAWINGS

FIG. 1 is a perspective view showing the outside of the drug delivery device.

FIG. 2 is a cross sectional view of the distal end of the drug delivery device.

FIG. 3 is a cross sectional view lengthwise through the device.

FIG. 4 is a cross sectional view of an alternative embodiment of the device

### DETAILED DESCRIPTION OF THE INVENTION

The invention comprises a heat source that is fluidly connected with a confined area containing a set of inert particles that are coated with a therapeutic agent, the confined area also being fluidly connected to an outlet through which a user can inhale the therapeutic agent after it becomes volatilized. Porous barriers separate the confined area containing the set of particles from the heat source and the outlet, so that the particles do not migrate from the confined area.

The invention further comprises a method of delivering a drug to the lungs of a user, wherein the user activates the heat source of a drug delivery device to volatilize a drug as described herein and applies suction to the mouthpiece of the device.

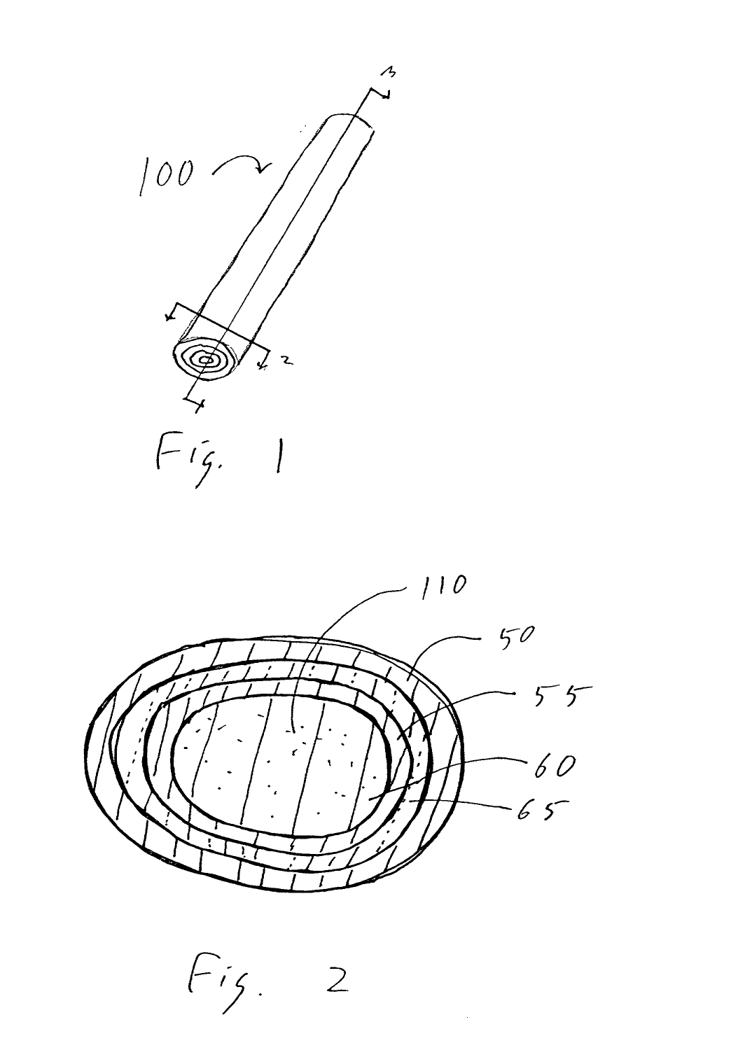

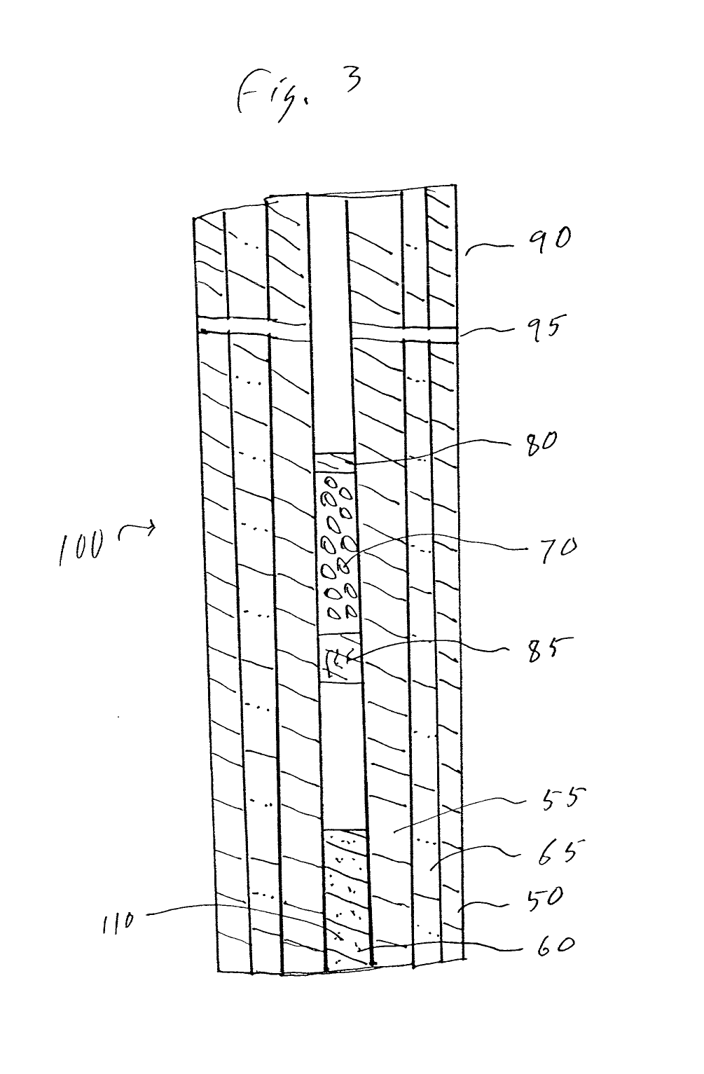

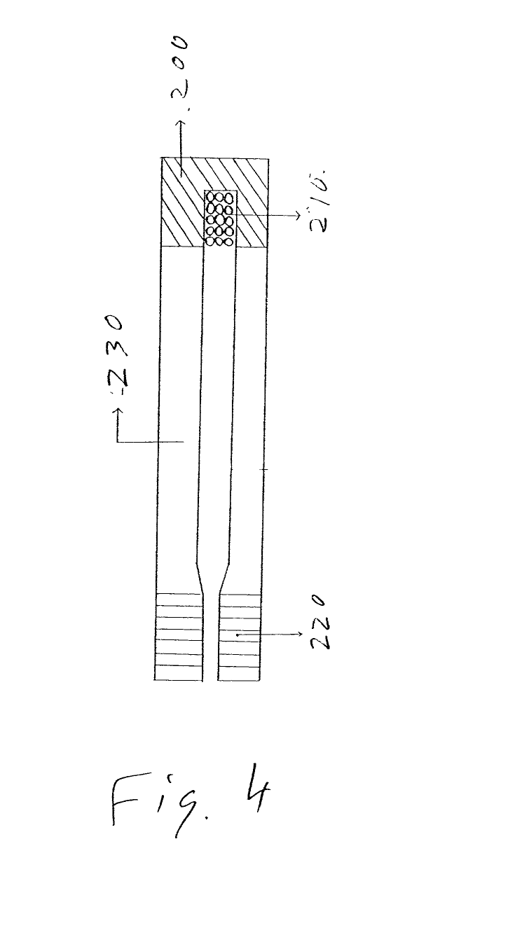

In one specific embodiment, as shown in FIG. 1, the device 100 appears as a graphite tube that is about 10 cm in length, having an oval shaped cross section. Preferably, the oval-shaped cross-section has a diameter of about 10 mm in its widest dimension, and a diameter of about 7 mm in its narrowest dimension. As shown in FIG. 2, device includes three layers running throughout the device, an outer graphite or aluminum layer 50, and inner graphite or aluminum layer, 55 surrounding a central passageway 60 and a layer of compacted glass fiber 65 between the inner and outer graphite or aluminum layers. The inner graphite or aluminum layer acts as an insulator, holding the heat source and providing conductive heat transfer. The outer layers provide additional insulation so that the device can be held by a user. The tip of the device incorporates a carbon heat source 110 filling the central passageway on the distal end section of the device. The carbon heat source comprises a mixture of powdered graphite and sodium carbopol, which is surrounded by an insulating compacted glass fiber coat 120. (Carbopol® is a registered trademark of B. F. Goodrich Corporation). The term carbopol as used herein refers to a polyvinyl polymer of acrylic acid which may be crosslinked with a polyallyl ether of sucrose or pentaerythritol. As shown schematically in cross section in FIG. 3 (not drawn to scale), the distal end of the device contains the heat source, the middle section contains the drug substance, which is in the form of thin coat on a core of α-alumina beads 70, to provide a large surface area for volatilization by the heated air. Plugs of porous glass fiber 80 and 85 are placed immediately on both sides of the drug-coated alumina spheres to stabilize the unit and prevent movement down the graphite tube. The device is terminated in a glass fiber mouthpiece 90 containing a concentric row of perforations 95 to facilitate air mixing with drug vapor. The patient activates the device, which can be called a Thermal Vaporizing Device or TVD, by igniting the tip. When the heat source is ignited, it heats the incoming air, which is then drawn through the device by the patient sucking on the mouthpiece. The resulting heated air is then drawn over the drug-coated alumina spheres causing immediate volatilization of the drug. The hot vaporized drug, in the form of very small liquid particles, passes through to the mouthpiece and into the lungs of the user. A porous carbon fiber plug may be provided in or before the mouthpiece to cool the drug. Also, the mouthpiece may be provided with holes to let in outside air to cool the vaporized drug particles.

An alternative embodiment is shown in FIG. 4 wherein the heat source 200 and the inert drug-coated particles 210 are both contained in the distal end of the device 300, with the heat source surrounding the particles. The remainder of the device is a hollow tube 230 of glass fiber with a filter tip mouthpiece 220.

The drug delivery device of the present invention can be used to delivery any drug that can be coated onto inert particles such as alumina particles, and that can be volatilized by heating the air surrounding the particles. As used herein, the term “drug” refers to any compound or composition for which delivery into the lungs of a user is desired, particularly, the term refers to a therapeutic agent. Examples of drugs useful in the present invention include, but are not limited to, the following:

dronabinol (delta-9-tetrahydrocannabinol) and related cannabinoids such as: (−)-delta-9-tetrahydrocannabinol, (+)-delta-9-tetrahydrocannabinol, and delta-8-tetrahydrocannabinol, cannabinol, cannabigerol, cannabicyclol, cannabielsoic acid and their respective pure enantiomers and/or diastereomers, combinations of the above cannabinoids, plant extracts containing any or all of the above cannabinoids, all naturally occurring cannabinoids, all therapeutically useful and pharmacologically active cannabinoids, and cannabinoid receptor antagonists, cannabinoid metabolites, all natural and synthetic non-psychoactive cannabinoids and their analogs (e.g. dexanabinol), and all psychoactive cannabinoids and their analogs (e.g. nantradol, nabitan);

volatilizable drugs (i.e., compounds preferably with a relatively low vapor pressure [boiling point 175-300° C.]) that are currently used to treat all acute and chronic manifestations of pain (e.g. opiates, nicotinic receptor antagonists), psychiatric disorders (such as psychosis, anxiety, and depression), sleep disorders, narcolepsy, epilepsy, seizure, electroconvulsive disorders, migraine, CNS degenerative disorders, diseases of cognitive function (e.g. Parkinson's syndrome, Alzheimer's disease, Huntington's chorea, ALS, Tourettes syndrome, tardive dyskinesia, hyperkinesia), mania, attention deficit disorder, schizophrenia, eating disorders, acute hypertension, multiple sclerosis, asthma (bronchodilators), drug and alcohol addiction, drug abuse, cardiovascular episodes (hypertension), anorexia (appetite stimulation), and emesis;

volatilizable therapeutic agents to treat emesis/nausea in patients undergoing cancer chemotherapy, and HIV patients receiving combination therapy, and to treat progressive anorexia and stimulate appetite in patients suffering from AIDS wasting or undergoing cancer chemotherapy;

volatilizable therapeutic agents to reduce intra-ocular pressure in patients suffering from glaucoma;

volatilizable therapeutic agents that act as neuroprotective agents in all brain trauma/stroke incidents, ischemia and all related neurological diseases and pathologies;

volatilizable therapeutic agents for the treatment of all spastic disorders, particularly in patients suffering from multiple sclerosis, and patients with spinal cord injuries;

volatilizable therapeutic agents for the treatment of movement disorders in dystonia, Huntington's chorea, Parkinson's syndrome, Tourette's syndrome;

volatilizable therapeutic agents for the treatment of alcohol and opiate withdrawal syndromes;

volatilizable chemotherapeutic agents with antibacterial, anti-infection, antiviral, and antifungal activity;

volatilizable agents for the treatment of motion sickness and related disorders such as vertigo;

volatilizable agents for the treatment of cough, and infections in the oral mucosal area;

volatilizable agents which have diagnostic applications in the lung;

volatilizable agents with local anesthetic properties;

volatilizable agents for the treatment of allergic reactions (e.g. antihistamines, steroids, non-steroidal anti-inflammatory agents); and

volatalizable vitamins and vitamin supplements.

The inert particles may be any particles that do not react covalently with organic materials such as drugs and that do not decompose in the temperature ranges of the invention. The preferred material for the inert particles is α-alumina with a particle size such that the plurality of the particles will have a large surface area for adsorbing the drug. Typically, the drug is coated onto the inert particles by dissolving the drug in a solvent, combining the drug solution with the inert particles and mixing so that the drug solution is thoroughly distributed among the inert particles, then evaporating the solvent so that the drug is left adhering to the particles. Typically, the solvent is selected to volatilize at a much lower temperature than that at which the drug volatilizes, so that the solvent can be removed without volatilizing the drug.

The heat source is preferably an ignitable material that burns with sufficient heat so that air that is drawn into the device is heated sufficiently to volatilize and desorb the drug from the surface of the inert particles. The preferred material is a carbonaceous material such as a mixture of powdered graphite and sodium carbopol, a polyvinyl polymer of acrylic acid crosslinked with a polyallyl ether of sucrose or pentaerythritol.( Carbopol® is a registered trademark of B. F. Goodrich Corporation). Other ignitable materials may be used. Further, other types of heat source may be used, such as chemical or electrical heat sources.

### EXAMPLE

In the following example, studies were carried out on the adsorption and desorption of Δ⁹-THC onto α-alumina particles with a target of fulfilling the following objectives:

a) Establishing a Δ⁹-THC loading procedure.

b) Determining the loading distribution of Δ⁹-THC.

c) Determining the loading amount of Δ⁹-THC.

d) Analysis of the loaded Δ⁹-THC material.

e) Desorption experiments with a simulated thermal vaporization device.

Loading Procedure and Loading Distribution

1. A weighed quantity of Δ⁹-THC was dissolved in absolute alcohol using one of the following suitable mixing devices: cyclomixer, sonicator, and/or hand-held mixer.

2. A weighed quantity of α-Alumina particles (100 mesh) were placed into a suitable container and the solution in step 1 was added in small volumes with intermittent mixing. Mixing was continued after each additional volume of the ethanolic solution of Δ⁹-THC was added to the α-Alumina particles, to ensure an even distribution of the drug solution before the next aliquot part of the drug solution was added and mixed.

3. After complete addition of the drug solution, the α-Alumina particles containing the adsorbed drug material were dried indirectly at 30-40° C. using a hot air gun until an ethanolic odor was not perceived.

4. The α-Alumina particles containing the adsorbed dried Δ⁹-THC was analyzed for drug content.

5. The loading distribution of the drug throughout the bulk α-Alumina particulate sample was determined from the analysis of 2-3 random samples obtained from different locations within the material in 4 above.

Determination of Loading Amount and Analysis of Loaded Drug Material

The loading amount of drug per 100 mg of α-Alumina adsorbent can be determined based on the final design requirements of the TVD. Quantification of loaded drug can be determined by the gas chromatographic (GC) method of analysis (see next section for details). The Δ⁹-THC loading amount was determined to be 0.85 mg per 100 mg α-Alumina.

Desorption Experiments

The objective of these experiments is to provide experimental proof that the loaded drug will desorb from the α-Alumina particles at a particular experimental temperature.

a) Delivery of Δ⁹-THC using an experimental apparatus that simulates a TVD device:

The Δ⁹-THC loaded α-Alumina particles were placed into a glass tube, and the tube then closed with a cotton cloth from both the ends; one end of the tube was connected to a paper filter cartridge/cold finger. A temperature probe (Type K thermocouple; Cole-Parmer) was inserted in-between the filter cartridge and the glass tube. The other end of the cartridge was connected to a vacuum pump. The amount of vacuum utilized was controlled through a regulator. The vacuum applied was just sufficient to fluidize the material in the glass tube. The glass tube was then externally heated with a hot air generator heat gun at a temperature of about 350-400° C. for about 15 min. and the equipment was then dismantled.

The following components of the above unit were analyzed by Gas Chromatography (GC):

1. Paper Filter Cartridge

2. Cloth filter from the cartridge/cold finger (top) end and bottom end

3. Glass tube

4. Spent α-Alumina particles

b) Thermo-Gravimetric Analysis(TGA) Experiments:

The objective of this method of analysis was to evaluate the desorption pattern of Δ⁹-THC from the α-Alumina particles. α-Alumina particles containing adsorbed Δ⁹-THC were subjected to TGA analysis. The coated alumina particles were heated at 350° C. for 10 minutes. Condensate collected on the lid of the TGA sample chamber was rinsed with a measured aliquot of solvent chloroform and the sample analyzed. The spent alumina particles were also rinsed with a measured aliquot of solvent chloroform and the sample analyzed. Both samples were analyzed for delta-9-THC content by gas chromatography. The results are set forth in Table 1 below

TABLE 1

| | | | |
|---|---|---|---|
| | Δ⁹-THC Content in | Δ⁹-THC Accounted | |
| | the coated-alumina | For in Sample By | Material |
| Sample ID | starting material | GC Analysis | Balance |

TVD Device Simulation Experiments that utilized
paper filter cartridge as ⁹-THC trap

| | | | |
|---|---|---|---|
| Paper filter and | 5.06 mg | 93.95 μg: Δ⁹-THC | 4.703 |
| cloth (top) from the | | | (92.9%) |
| filter end | | | |
| Glass tube | | 90.55μg: ⁹-THC | |
| Spent α-Alumina | | 172.8 μg: ⁹-THC | |
| particles | | | |

TVD Device Simulation Experiments that
utilized cold finger as ⁹-THC trap

| | | | |
|---|---|---|---|
| Cold Finger | 5.00 mg | 0 μg: Δ⁹-THC | 4.646 |
| | | | (92.9%) |
| Cloth filter-top | | 157.30 μg: Δ⁹-THC | |
| Cloth filter- | | 0 μg: Δ⁹-THC | |
| bottom | | | |
| Glass tube | | 112.15 μg: Δ⁹-THC | |
| Spent α-Alumina | | 84.21 μg: Δ⁹-THC | |
| particles | | 3.92 μg: Cannabinol | |

TGA Experiment

| | | | |
|---|---|---|---|
| Condensate | 3.25 mg | 254.94 μg | 3.00 |
| collected on the lid | | | (92.3%) |
| of the TGA sample | | | |
| chamber | | | |

Conclusions

1. Δ⁹-THC can be uniformly coated on α-alumina particles.

2. It is evident from the simulated TVD experiments and TGA experiment that Δ⁹-THC coated α-alumina particles can be desorped of Δ⁹-THC utilizing forced heated air or direct heat at the appropriate temperature.

Obviously, many modifications and variations of the present invention are possible in light of the above teachings. It is therefore to be understood that, within the scope of the appended claims, the invention may be practiced otherwise than as specifically described.

We claim:

1. A drug delivery device for delivery of a drug to the lungs of a user, the drug delivery device comprising
a plurality of inert inorganic particles, the particles having the drug adsorbed thereon,
means for heating the plurality of inert inorganic particles to cause the drug to volatilize and become desorbed from the plurality of inert inorganic particles, and
means for directing the volatilized and desorbed drug into the lungs of the user.

2. The drug delivery device of claim 1 wherein the means for heating the plurality of inert inorganic particles comprises an ignitable carbonaceous material that, when ignited, heats air that is then drawn into fluid contact with the plurality of inert inorganic particles.

3. The drug delivery device of claim 2 wherein the ignitable carbonaceous material is a mixture of powdered graphite and sodium carbopol.

4. The drug delivery device of claim 1 wherein the plurality of inert inorganic particles are made of alumina.

5. The drug delivery device of claim 1 wherein the plurality of inert inorganic particles are 100 mesh α-alumina particles.

6. The drug delivery device of claim 1 wherein the plurality of inert inorganic particles are contained in a chamber and wherein means for directing the volatilized and desorbed drug into the lungs of the user is by a mouthpiece that is fluidly connected to the chamber containing the inert organic particles, so that the volatilized and desorbed drug can be drawn into the lungs of the user by suction.

7. The drug delivery device of claim 6 further comprising a porous barrier between the mouthpiece and the chamber to prevent the inert organic particles from being drawn into the lungs of the user.

8. The drug delivery device of claim 7 wherein the porous barrier is made of porous glass fiber.

9. A drug delivery device for delivery of a drug to the lungs of a user, the drug delivery device comprising
a tubular housing having
a proximal end section comprising a mouthpiece,
a distal end section comprising a heat source that can be selectively activated, and
a middle section between the proximal end section and the distal end section,
the middle section having an chamber containing a plurality of inert inorganic particles, the particles having the drug adsorbed as a coating thereon,
wherein the heat source of the distal end section, the plurality of inert inorganic particles in the middle section and the mouthpiece of the proximal end section are fluidly connected through an interior passageway of the tubular housing and wherein the distal end section is fluidly connected to outside air and wherein the proximal end section can be connected to the mouth of a user so that air can be drawn into the heat source by suction applied by the user and heated and wherein the heated air can be drawn into the chamber of the middle section to heat, volatilize and desorb the drug from the inert particles and wherein the volatilized and desorbed drug can be drawn from the chamber of the middle section into the mouthpiece and then into the mouth and lungs of the user.

10. The drug delivery device of claim 9 wherein the heat source is an ignitable carbonaceous material.

11. The drug delivery device of claim 10 wherein the ignitable carbonaceous material is a mixture of powdered graphite and sodium carbopol.

12. The drug delivery device of claim 9 wherein the plurality of inert inorganic particles are made of alumina.

13. The drug delivery device of claim 9 wherein the plurality of inert inorganic particles are 100 mesh α-alumina particles.

14. The drug delivery device of claim 9 wherein the tubular member further comprises insulating material to insulate the heat source, interior passageway, and chamber from fingers of a user.

15. The drug delivery device of claim 9 further comprising a porous barrier between the distal end section and the chamber that prevents the plurality of inert inorganic particles from escaping from the chamber into the distal end section.

16. The drug delivery device of claim 9 further comprising a porous barrier between the proximal end section and the chamber that prevents the plurality of inert inorganic particles from escaping from the chamber into the proximal end section.

17. The drug delivery device of claim 9 wherein the mouthpiece contains a plurality of perforations that fluidly communicate from the outside air into the interior passageway of the mouthpiece to allow air to drawn into the lungs of the user along with the volatilized and desorbed drug.

18. A method of delivering a drug to the lungs of a user, the method comprising the steps of
providing a drug delivery device comprising a plurality of inert inorganic particles, the particles having the drug adsorbed thereon, means for heating the plurality of inert inorganic particles to cause the drug to volatilize and become desorbed from the plurality of inert inorganic particles, and means for directing the volatilized and desorbed drug into the lungs of the user,
heating the plurality of inert inorganic particles to cause the drug to volatilize and become desorbed from the plurality of inert inorganic particles, and
directing the volatilized and desorbed drug into the lungs of the user.

19. The method of claim 18 wherein the means for heating the plurality of inert inorganic particles comprises an ignitable carbonaceous material that, when ignited, heats air that is then drawn into fluid contact with the plurality of inert inorganic particles, and wherein the step of heating the plurality of inert inorganic particles is carried out by heating the carbonaceous material.

20. The method of claim 18 wherein the plurality of inert inorganic particles are contained in a chamber and wherein means for directing the volatilized and desorbed drug into the lungs of the user is by a mouthpiece that is fluidly connected to the chamber containing the inert organic particles, and wherein the step of directing the volatilized and desorbed drug into the lungs of the user is carried out by having the user apply suction to the mouthpiece.

21. A method of delivering a drug to the lungs of a user, the method comprising the steps of
providing a drug delivery device comprising a tubular housing having a proximal end section comprising a mouthpiece, a distal end section comprising a heat source that can be selectively activated, and a middle section between the proximal end section and the distal end section, the middle section having an chamber containing a plurality of inert inorganic particles, the particles having the drug adsorbed as a coating thereon, wherein the heat source of the distal end section, the plurality of inert inorganic particles in the middle section and the mouthpiece of the proximal end section are fluidly connected through an interior passageway of the tubular housing and wherein the distal end section is fluidly connected to outside air and wherein the proximal end section can be connected to the mouth of a user so that air can be drawn into the heat source by suction applied by the user and heated and wherein the heated air can be drawn into the chamber of the middle section to heat, volatilize and desorb the drug from the inert particles and wherein the volatilized and desorbed drug can be drawn from the chamber of the middle section into the mouthpiece and then into the mouth and lungs of the user,
activating the heat source, and
allowing the user to apply suction to the mouthpiece.

22. The drug delivery device of claim 1 wherein the drug is selected from the group consisting of:
dronabinol (delta-9-tetrahydrocannabinol), (−)-delta-9-tetrahydrocannabinol, (+)-delta-9-tetrahydrocannabinol, and delta-8-tetrahydrocannabinol, cannabinol, cannabigerol, cannabicyclol, cannabielsoic acid and their respective pure enantiomers and/or diastereomers, combinations of the above cannabinoids, plant extracts containing any or all of the above cannabinoids, all naturally occurring cannabinoids, all therapeutically useful and pharmacologically active cannabinoids, and cannabinoid receptor antagonists, cannabinoid metabolites, all natural and synthetic non-psychoactive cannabinoids and their analogs (e.g. dexanabinol), and all psychoactive cannabinoids and their analogs (e.g. nantradol, nabitan);
volatilizable drugs (i.e., compounds preferably with a relatively low vapor pressure [boiling point 175-300° C.]) that are currently used to treat all acute and chronic manifestations of pain (e.g. opiates, nicotinic receptor antagonists), psychiatric disorders (such as psychosis, anxiety, and depression), sleep disorders, narcolepsy, epilepsy, seizure, electroconvulsive disorders, migraine, CNS degenerative disorders, diseases of cognitive function (e.g. Parkinson's syndrome, Alzheimer's disease, Huntington's chorea, ALS, Tourettes syndrome, tardive dyskinesia, hyperkinesia), mania, attention deficit disorder, schizophrenia, eating disorders, acute hypertension, multiple sclerosis, asthma (bronchodilators), drug and alcohol addiction, drug abuse, cardiovascular episodes (hypertension), anorexia (appetite stimulation), and emesis;
volatilizable therapeutic agents to treat emesis/nausea in patients undergoing cancer chemotherapy, and HIV patients receiving combination therapy, and to treat progressive anorexia and stimulate appetite in patients suffering from AIDS wasting or undergoing cancer chemotherapy;
volatilizable therapeutic agents to reduce intra-ocular pressure in patients suffering from glaucoma;
volatilizable therapeutic agents that act as neuroprotective agents in all brain trauma/stroke incidents, ischemia and all related neurological diseases and pathologies;
volatilizable therapeutic agents for the treatment of all spastic disorders, particularly in patients suffering from multiple sclerosis, and patients with spinal cord injuries;
volatilizable therapeutic agents for the treatment of movement disorders in dystonia, Huntington's chorea, Parkinson's syndrome, Tourette's syndrome;
volatilizable therapeutic agents for the treatment of alcohol and opiate withdrawal syndromes;
volatilizable chemotherapeutic agents with antibacterial, anti-infection, antiviral, and antifungal activity;
volatilizable agents for the treatment of motion sickness and related disorders such as vertigo;
volatilizable agents for the treatment of cough, and infections in the oral mucosal area;
volatilizable agents which have diagnostic applications in the lung;
volatilizable agents with local anesthetic properties;
volatilizable agents for the treatment of allergic reactions (e.g. antihistamines, steroids, non-steroidal anti-inflammatory agents); and
volatalizable vitamins and vitamin supplements.
